(19) United States
(12) Patent Application Publication
(10) Pub. No.: US 2007/0250003 A1
(43) Pub. Date: Oct. 25, 2007

(54) Fluid activated retractable safety syringe

(76) Inventors: Rex O. Bare, Lake Forest, CA (US); Robert D. Miller, Costa Mesa, CA (US)

Correspondence Address: STETINA BRUNDA GARRED & BRUCKER, 75 ENTERPRISE, SUITE 250, ALISO VIEJO, CA, 92656, US

(21) Appl. No.: 11/729,100

(22) Filed: Mar. 28, 2007

Related U.S. Application Data
(60) Provisional application No. 60/788,800, filed on Apr. 3, 2006.

(51) Int. Cl. A61M 5/00

(52) U.S. Cl. 604/110

(57) ABSTRACT

A retractable safety syringe is provided wherein a retraction force retracts a needle and a needle holder into a syringe body when a piston engages the needle holder. The engagement between the piston and the needle holder may be fluid activated such that engagement between the piston and needle holder exists only when fluid is in a variable fluid chamber. In particular, surface tension from the fluid on an annular suction groove or pocket and a textured top surface of the needle holder creates a suction force applied to the needle holder. The retraction force acts on the needle holder via the suction force to retract the needle holder and needle into the syringe body.

### CROSS-REFERENCE TO RELATED APPLICATIONS

This application claims the benefits of provisional patent application Ser. No. 60/788,800, filed Apr. 3, 2006, the entire content of which is incorporated herein by reference.

### STATEMENT RE: FEDERALLY SPONSORED RESEARCH/DEVELOPMENT

Not Applicable

### BACKGROUND

The present invention relates generally to a retractable safety syringe for injecting a patient with medication.

Currently, there are over 250 different types of retractable safety syringes. These safety syringes prevent accidental needle reuse of previously used safety syringes and accidental needle prickings during the administration of medication by retracting a needle of the syringe into the syringe body after medication administration.

Safety syringes may be typically provided to medical professionals without medication such that the medical professional can fill the selected medication into a variable fluid chamber of the syringe and administer the medication to the patient. This is a two step process, specifically (1) filling a variable fluid chamber with medication and (2) injecting the medication into the patient. During the first step, a piston of the syringe is disposed adjacent to a needle holder but does not engage the needle holder. The piston is retracted to fill the variable fluid chamber with fluidic medication. In contrast, during the second step, the piston is traversed toward the needle holder to inject the fluidic medication into the patient. At the end of the piston's stroke, the piston of the syringe is disposed adjacent to the needle holder and engages the needle holder. After the piston engages the needle holder, the piston, needle holder and needle are retracted into the syringe body. As such, the piston does not engage the needle holder during the first step but does engage the needle holder during the second step.

Prior art methods of preventing engagement between the piston and needle holder during the first step and ensuring engagement therebetween during the second step exists. Unfortunately, the current methods of preventing engagement during the first step and ensuring engagement during the second step are unsatisfactory.

Accordingly, there is a need in the art for an improved retractable safety syringe.

### BRIEF SUMMARY

The present invention addresses the problems discussed above, discussed below and those that are known in the art.

A safety syringe is provided wherein engagement between a piston and needle holder is accomplished via a fluid activated suction force. In particular, the piston may have an annular suction groove. Also, the needle holder may have a textured top surface which is sized and configured to mate with the annular suction groove. When a variable fluid chamber is dry (i.e., no fluid in the variable fluid chamber), the annular suction groove does not create a suction force on the textured top surface upon contact because the texture of the top surface permits air to flow into the annular suction groove when the piston is drawn away from the needle holder.

In contrast, when the variable fluid chamber is filled with fluidic medication, the annular suction groove creates a suction force on the textured top surface upon contact because surface tension of the fluid on the annular suction groove and the textured top surface seals the annular suction groove onto the textured top surface. No air is permitted to enter the annular suction groove when the piston is drawn away from the needle holder. The suction force draws the needle holder and needle into the syringe body when the piston is drawn toward a proximal end of the syringe.

### BRIEF DESCRIPTION OF THE DRAWINGS

These and other features and advantages of the various embodiments disclosed herein will be better understood with respect to the following description and drawings, in which like numbers refer to like parts throughout, and in which:

FIG. 1 is a front cross-sectional view of a safety syringe of a first embodiment in a retracted position illustrating a piston having an annular suction groove and a needle holder having a textured top surface;

FIG. 2 is a bottom view of the annular suction groove of a piston seal of FIG. 1;

FIG. 2a is a cross sectional view of the piston seal of FIG. 2;

FIG. 2b is a bottom perspective view of the piston seal of FIG. 2;

FIG. 3 is a top view of the textured top surface of the needle holder of FIG. 1;

FIG. 3a is a cross sectional view of the needle holder of FIG. 3;

FIG. 3b is a top perspective view of the needle holder of FIG. 3;

FIG. 4 is a front cross-sectional view of the safety syringe of FIG. 1 wherein the piston is in an extended position;

FIG. 5 is a front cross-sectional view of the safety syringe of FIG. 1 with the needle and needle holder retracted into the safety syringe and the needle canted toward one side or against the inner surface of the body;

FIG. 6 is a front cross sectional view of a safety syringe of a second embodiment in a retracted position illustrating a needle holder removeably engaged to the body via a retaining member;

FIG. 7 is an enlarged view of the needle holder shown in FIG. 6;

FIG. 8 is a front cross sectional view of the safety syringe shown in FIG. 6 with a piston in a second extended position;

FIG. 9 is a front cross sectional view of the safety syringe shown in FIG. 6 with the piston engaged to the needle holder and the piston in the retracted position;

FIG. 10 is an enlarged view of a piston seal;

FIG. 11 is a front cross sectional view of the safety syringe with the piston in a first extended position;

FIG. 12 is a top view of a braking mechanism; and

FIG. 13 is a front cross sectional view of a safety syringe with a spring mechanism.

### DETAILED DESCRIPTION

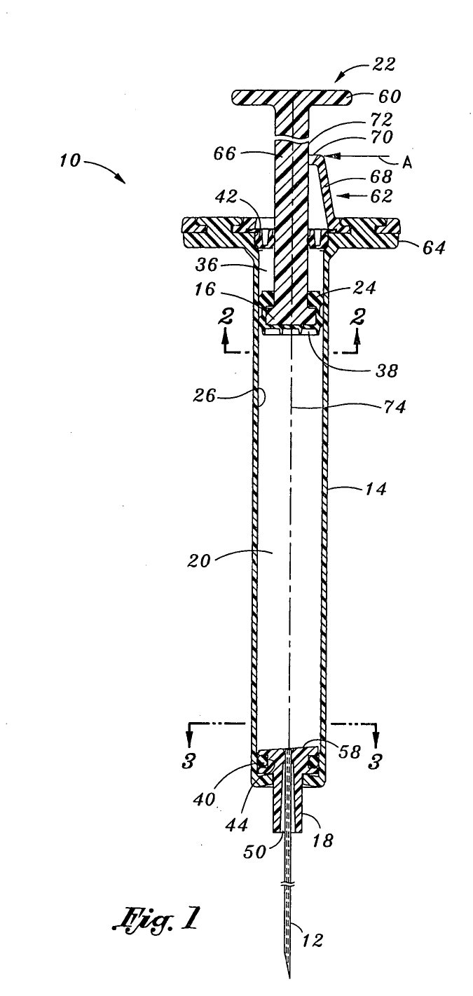

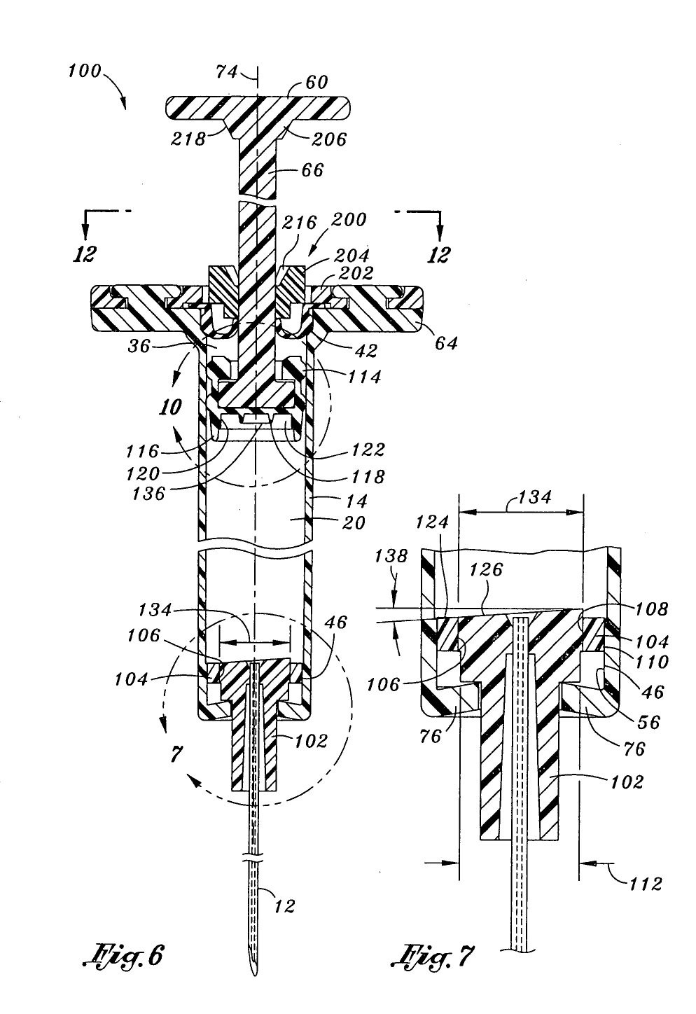

Referring now to the drawings, which are for the purposes of illustrating the preferred embodiments of the safety syringe 10 and not for the purpose of limiting the same, FIGS. 1, 4 and 5 are a front cross-sectional view of the safety syringe 10 of a first embodiment, and FIGS. 6-9 and 11 are a front cross-sectional view of the safety syringe 100 of a second embodiment. The safety syringe 10 shown in FIG. 1 mitigates against accidental reuse of previously used needles and accidental needle pricking from contaminated needles. In particular, after the safety syringe 10 has been used to inject fluidic medication into a patient, the needle 12 retracts into a body 14 of the safety syringe 10 immediately after administration of the medication. The retraction of the needle 12 into the body 14 of the safety syringe 10 is accomplished via a suction force created at an interface between a piston 16 and a needle holder 18. The suction force exists or is activated when fluid (e.g., medication, and the like, etc.) is filled in a variable fluid chamber 20 of the safety syringe 10. Accordingly, the piston 16 does not engage the ferrule until the variable fluid chamber 20 has been filled with fluidic medication and after the medication has been administered to the patient.

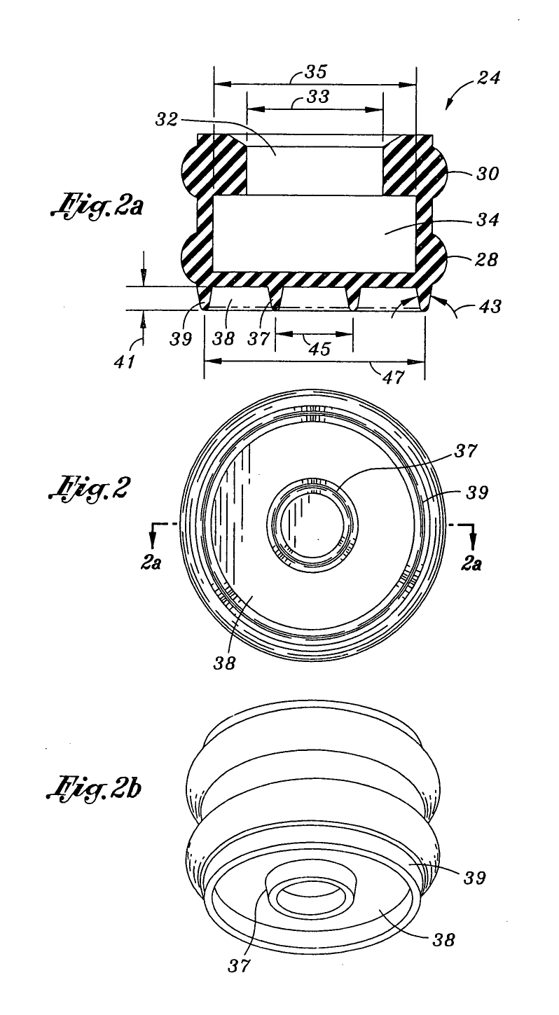

The body 14 may have a plunger 22 partially disposed therewithin which extends out of a proximal end of the body 14. The plunger 22 may have a piston 16 disposed within the syringe body 14. The piston 16 may have a molded piston seal 24 disposed entirely around the piston 16, as shown in FIG. 1. FIG. 2 is a bottom view of the piston seal 24. FIG. 2a is a cross sectional view of the piston seal 24. FIG. 2b is a perspective view of the piston seal 24. The piston seal 24 provides a fluid tight interface between the piston 16 and an inner surface 26 of the body 14. Lateral sides of the piston seal 24 may have a lower annular ring 28 and an upper annular ring 30 (see FIG. 2a) which engages the entire inner circumference of the syringe body 14 to provide the fluid tight interface. The top surface of the piston seal 24 may have a central aperture 32 (see FIG. 2a) through which the piston 16 is inserted. The central aperture 32 may have a diameter 33 of about 0.175 inches. The central aperture 32 may also lead to a central cavity 34 of the piston seal 24 in which the piston 16 itself resides. The central cavity 34 may have a diameter 35 of about 0.260 inches.

During assembly, the piston seal 24 being made from a generally stretchable resilient and flexible material may be disposed over the piston 16. By way of example and not limitation, the piston seal 24 may have a hardness of about twenty (20) to fifty (50) on a shore A scale. The piston 16 and the piston seal 24 are inserted into the body 14 of the safety syringe 10. Also, an interference fit exists between the body 14, piston seal 24 and piston 16 such that the annular rings 28, 30 push against the inner surface 26 of the body 14 forming a fluid tight interfaceface therebetween. In this manner, fluid filled in the variable fluid chamber 20 does not pass into a variable vacuum compartment 36. Also, as will be discussed below, air molecules within the variable fluid chamber 20 does not pass into the variable vacuum compartment 36.

An annular suction groove 38 may be formed on a lower distal surface of the piston seal 24, as shown in FIGS. 2, 2a, and 2b. An inner ring 37 may have a diameter 45 of about 0.100 inches. An outer ring 39 may have a diameter 47 of about 0.283 inches. A depth 41 of the annular suction groove 38 may be about 0.030 inches. The inner and outer rings 37, 39 may have an angled tip 43 of about twenty (20) degrees. The annular suction groove 38, as will be further discussed below, engages the needle holder 18 in the presence of fluid and does not engage the needle holder 18 in the absence of fluid.

The variable fluid chamber 20 is defined by the volume between the piston seal 24 and a first seal 40 disposed at the distal end of the body 14. As the piston 16 moves toward a retracted position and an extended position, the volume of the variable fluid chamber 20 varies. Similarly, the variable vacuum compartment 36 which is defined by volume between the piston seal 24 and a second seal 42 disposed at a proximal end of the body 14 also has a volume which conversely varies with respect to the volume of the variable fluid chamber 20 as the piston 16 is traversed toward the retracted position and the extended position. As used in the first embodiment, the extended position is when the piston contacts the needle holder. Generally, the retracted position is when the piston is closer to the proximal end of the body compared to the distal end. But, the retracted position may include the situations when the piston does not contact the needle holder and the piston is closer to the distal end of the body compared to the proximal end.

The first seal 40 may be disposed about a ring groove 44 of the needle holder 18. The first seal 40 provides an interference fit between the needle holder 18 and an inner surface of a raised step 46 (see FIG. 5) of the syringe body 14. In this manner, a fluid tight interface is created between the needle holder 18 and the syringe body 14 such that medication or fluid does not leak out of the syringe body 14 through its distal end. In review, the variable fluid chamber 20 forms a fluid tight volume in which medication or fluid is filled and injected into the patient through the needle 12.

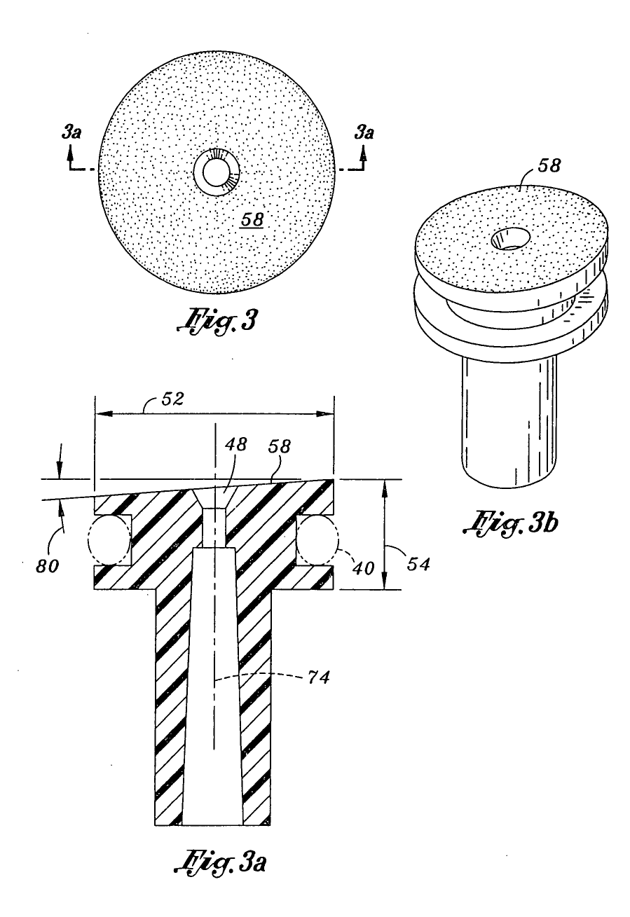

The needle 12 is disposed within a central aperture 48 of the needle holder 18 (see FIG. 3a). A distal portion of the needle holder 18 has a gap 50 (see FIG. 1) between the central aperture 48 of the needle holder 18 and the needle 12 itself. After the needle 12 is disposed within the central aperture 48, adhesive is filled within the gap 50 to permanently retain the needle 12 on the needle holder 18.

The needle holder 18 and needle 12 may be temporarily engaged to the distal end of the body 14 via friction. In particular, an outer circumference 52 (see FIG. 3a) of an upper portion 54 of the needle holder 18 has an outer diameter (e.g., about 0.310 inches, etc.) which is slightly larger than an inner diameter 56 (see FIG. 5) of the raised step 46. The interference between the outer surface of the upper portion 54 and the raised step 46 creates a frictional force which retains the needle holder 18 to the distal end of the syringe body 14 or the inner surface of the raised step 46 until the piston 16 or plunger 22 engages the needle holder 18 to retract the needle holder 18 and needle 12 into the syringe body 14.

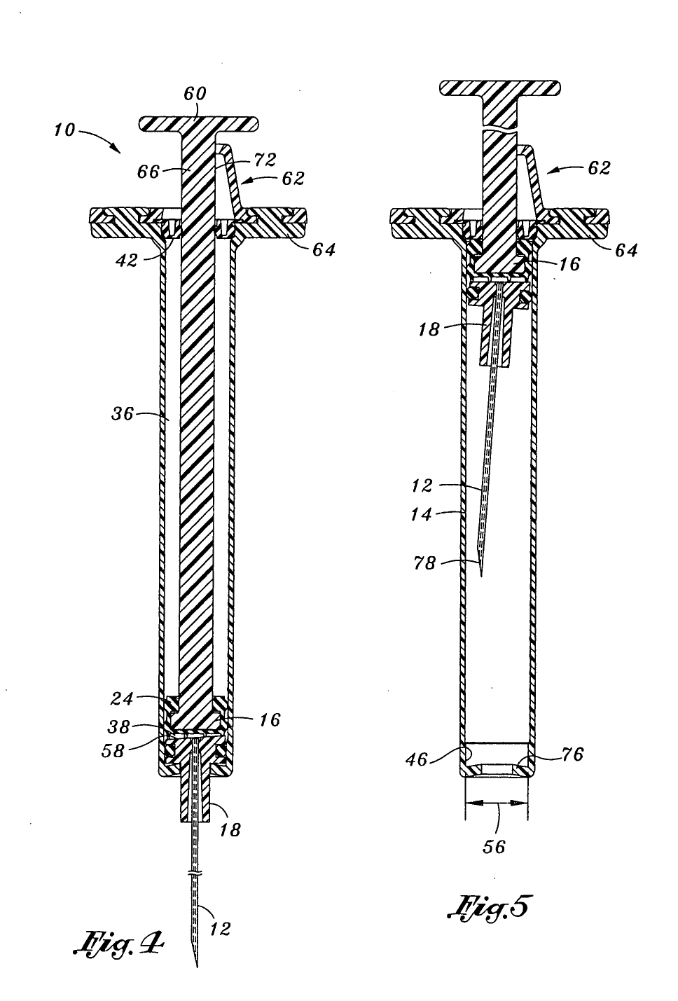

The variable vacuum compartment 36 defined by the volume within the syringe body 14 between the second seal 42 and the piston seal 24 is an airtight compartment and a fluid tight compartment. In particular, air molecules are not permitted to enter the variable vacuum compartment 36 by bypassing the second seal 42 or the piston seal 24. Accordingly, when the piston 16 is traversed from the retracted position toward the extended position, there is no corresponding influx of air molecules into the variable vacuum compartment 36. As a result, the variable vacuum compartment 36 produces a retraction force which urges the piston 16 back toward the retracted position. As the piston 16 is further traversed toward the extended position, the retraction force increases. When the piston 16 contacts or touches a top surface 58 of the needle holder 18 (see FIG. 4), the retraction force is greater than the friction force between the needle holder 18 and the raised step 46. If the piston 16 were to engage the needle holder 18, then the needle holder 18 and needle 12 would retract into the syringe body 14. Fortunately, during the operation of the syringe body 14, the piston 16 does not engage the needle holder 18 when the piston 16 is initially being traversed from the retracted position to the extended position to fill the variable fluid chamber 20 with medication because the engagement therebetween is fluid activated and no fluid is initially present in variable fluid chamber 20. Please note, that the safety syringe 10 may be provided to medical professionals without medication filled within the variable fluid chamber 20. When the piston 16 initially contacts the needle holder 18, no fluid or medication is contained or filled within the variable fluid chamber 20.

To fill the variable fluid chamber 20 with fluid or medication, the medical professional traverses the piston 16 to the extended position (see FIG. 4). Thereafter, the medical professional may insert the needle 12 into a medication container and traverse the piston 16 from the extended position to the retracted position (see FIG. 1) which then fills the variable fluid chamber 20 with medication or fluid. After the variable fluid chamber 20 is filled with an appropriate amount of medication, the medical professional may then insert the needle 12 into a patient and depress a thumb platform 60 to traverse the piston 16 from the retracted position to the extended position (see FIG. 4). The medical professional will depress the thumb platform 60 fully until the piston 16 contacts the needle holder 18 to eject as much of the medication out of the syringe body 14 and into the patient as possible. Since the variable fluid chamber 20 has been filled with fluid (i.e., fluidic medication), the piston 16 and the needle holder 18 are engaged to each other, as will be discussed further below.

The bottom surface of the piston seal 24 has an annular suction groove 38, as shown in FIGS. 2, 2a, and 2b. If the annular suction groove 38 was placed against a smooth flat surface, then the annular suction groove 38 would create a suction force on the smooth flat surface even if there were no fluid therebetween. However, the annular suction groove 38 is placed against a textured top surface 58 (see FIG. 3 and 3b) of the needle holder 18. As such, when the variable fluid chamber 20 is dry, then piston 16 does not engage the needle holder 18 via a suction force created by the annular suction groove 38. In contrast, when the variable fluid chamber 36 contains fluid, then the piston 16 does engage the needle holder 18 via a suction force created by the annular suction groove 38. Accordingly, when the piston 16 is traversed to the extended position to fill the variable fluid chamber 20 with medication, the piston 16 does not engage the needle holder 18 and retract the needle holder 18 and needle 12 into the syringe body 14 as the piston 16 is subsequently retracted toward the retracted position. The reason is that the syringe 10 is provided to the medical professional without any fluid contained within the variable fluid chamber 20.

When the medication has been filled into the variable fluid chamber 20 and the medication is administered to the patient by traversing the piston 16 to the extended position, the annular suction groove 38 creates a suction force on the textured top surface 58 of the needle holder 18 because surface tension of the fluid forms or completes the seal between the annular suction groove 38 and the textured top surface 58 of the needle holder 18 such that air molecules or fluid molecules are not permitted to enter the annular suction groove 38 thereby maintaining the suction force. Accordingly, after the medication has been injected into the patient, and the piston 16 contacts the needle holder 18, the annular suction 38 groove creates a suction force which is applied to the top surface 58 of the needle holder 18 as a result of the surface tension formed between the annular suction groove 38 and the textured top surface 58 of the needle holder 18.

The textured top surface 58 may be similar to #MT1055-4 fabricated by Mold Tech. More broadly, the textured top surface 58 may have a roughness which permits the annular suction groove 38 to produce a suction force on the top surface 58 of the needle holder 18 sufficient to draw the needle holder 18 into the syringe body 14 when fluid is present in the variable fluid chamber 20. Also, the textured top surface 58 may have a roughness which does not permit the annular suction groove 38 to produce the suction force on the top surface 58 of the needle holder 18 sufficient to draw the needle holder 18 into the syringe body 14 when fluid is not present in the variable fluid chamber 20.

In use, the safety syringe 10 is provided to the medical professional or user with the piston 16 in a retracted position (see FIG. 1). When medication is to be administered to a patient, the medical professional pushes down on the thumb platform 60 to traverse the piston 16 from the retracted position toward the extended position (see FIG. 4). The second seal 42 and the piston seal 24 forms an airtight compartment such that additional air molecules are not introduced to the variable vacuum compartment 36 as the piston 16 is traversed toward the extended position. This creates a retraction force which urges the piston 16 back toward the retracted position. As a result, the medical professional should not release the thumb platform 60 with his or her thumb because the piston 16 may immediately retract to the retracted position. Instead, when the piston is traversed to the extended position, the medical profession should maintain pressure on the thumb platform 60 and insert syringe's needle 12 into a medication container.

When the piston 16 is traversed to the extended position, the piston 16 may contact the top surface 58 of the needle holder 18. Fortunately, as discussed above, the annular suction groove 38 of the piston seal 24 does not create a suction force on the textured top surface 58 of the needle holder 18 so as to retract the needle 12 and needle holder 18 into the syringe body 14 when the piston 16 is subsequently traversed to the retracted position.

After the medical professional inserts the needle 12 into the medication container filled with fluidic medication,the medical professional may slowly release or balance the thumb pressure on the thumb platform 60 with the retraction force of the variable vacuum compartment 36 to slowly traverse the piston 16 from the extended position toward the retracted position. Such retraction of the piston 16 toward the retracted position fills the variable fluid chamber 20 with the medication. Now, the variable fluid chamber 20 is filled with fluid which contacts the textured top surface 58 of the needle holder 18 and the annular suction groove 38.

The medical professional removes the needle 12 from the medication container and inverts the safety syringe 10 to point the needle 12 upward. The medical professional or user then slightly depresses the thumb platform 60 thereby slightly traversing the piston 16 toward the extended position to remove any residual air within the needle 12 and the variable fluid chamber 20. The medical professional maintains pressure on the thumb platform 60 such that the piston 16 does not retract back toward the retracted position and readmit air within the needle 12 and the variable fluid chamber 20.

The medical professional or user may then inject the patient by depressing the thumb platform 60 fully toward the proximal end of the body 14 to thereby traverse the piston 16 from the retracted position to the extended position (see FIG. 4). When the thumb platform 60 is fully depressed, the bottom surface or the annular suction groove 38 contacts the top surface 58 of the needle holder 18. The surface tension creates or completes the seal between the annular suction groove 38 and the top textured surface 58 of the needle holder 18 to create a suction force. The retraction force of the variable vacuum compartment 36, being greater than the frictional force between the needle holder 18 and the raised step 46 retracts the needle holder 18 and needle 12 into the syringe body 14, as shown in FIG. 5. Since there is fluid within the variable fluid chamber 20 when the piston 16 contacts the needle holder 18, the fluid creates surface tension on the annular suction groove 38 and the top surface 58 of the needle holder 18 such that the annular suction groove 38 creates a suction force on the top surface 58 of the needle holder 18 to retract the needle holder 18 into the syringe body 14.

When the needle holder 18 traverses past the raised step 46, the upper portion 54 of the needle holder 18 no longer frictionally engages the syringe body 14 and is permitted to freely retract into the syringe body 14 via the retraction force of the variable vacuum compartment 36.

In the second embodiment of the safety syringe 100, the same is shown in FIGS. 6-11. The second embodiment of the safety syringe 100 also mitigates against accidental reuse of previously used needles and accidental needle prickings from contaminated needles in a similar manner compared to the safety syringe 10 of the first embodiment discussed above, namely, retracting the needle 12 into the body 14 after use. One difference between the second embodiment of the safety syringe 100 and the first embodiment of the safety syringe 10 is in the manner that the needle holder 102 is frictionally engaged to a distal end of the body 14. In the first embodiment of the safety syringe 10, the needle holder 18 and needle 12 may be temporarily engaged to the distal end of the body 14 via friction between the outer circumference 52 of the upper portion 54 of the needle holder 18 and the inner diameter 56 of the raised step 46, as discussed above. In the second embodiment of the safety syringe 100, the outer circumference 106 of the needle holder does not directly contact the inner diameter 56 of the raised step 46. Rather, when the needle holder 102 is disposed at the distal end of the body 14, a retaining member 104 is interposed between the needle holder 102 and the raised step 46. The retaining member 104 may have annular configuration which frictionally engages the outer circumference 106 of the needle holder 102 and the inner diameter 56 of the raised step 46. The raised step 46 is more clearly shown in FIGS. 6, 7 and 9. The retaining member 104 may have a square cross-sectional configuration and have an inner surface 108 and an outer surface 110, as shown in FIG. 7. The inner surface 108 of the retaining member 104 may frictionally engage the outer circumference 106 (see FIG. 7) of the needle holder 102. Also, the outer surface 110 of the retaining member 104 may frictionally engage the inner diameter 56 (see FIG. 9) of the raised step 46.

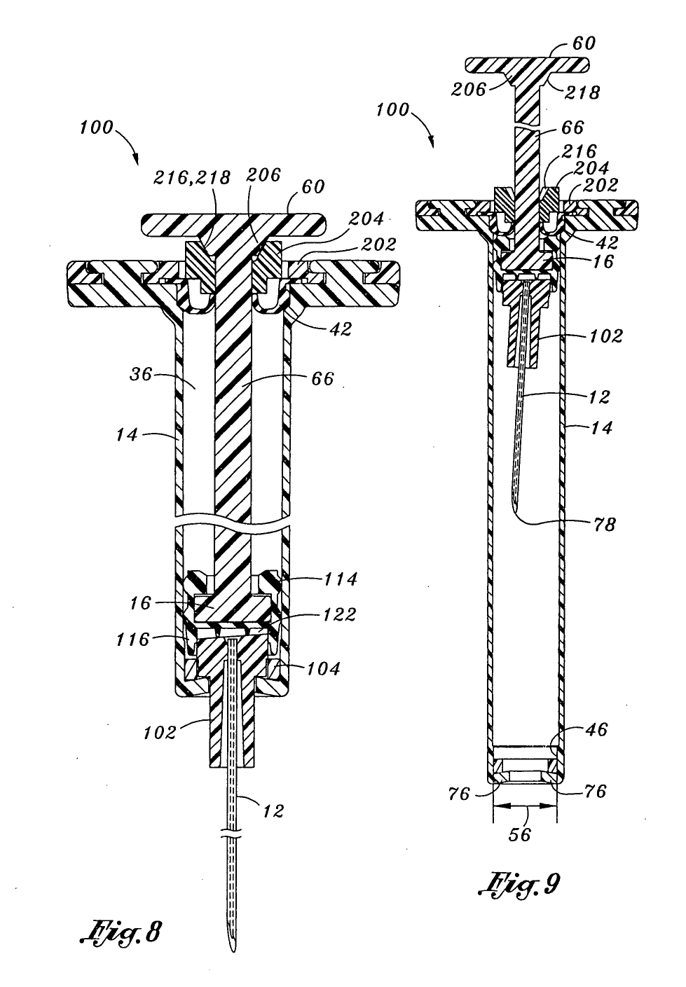

During operation of the safety syringe 100, the retaining member 104 may be displaced off of the outer circumference 106 of the needle holder 102 and about a reduced diameter 112 (see FIG. 7) of the needle holder 102, as shown in FIG. 8. When the retaining member 104 is displaced about the reduced diameter 112 of the needle holder 102 (see FIG. 8), the retaining member 104 releases the needle holder 102 such that the needle holder 102 and needle 12 may be retracted into the body 14 of the safety syringe 100, as discussed above in relation to the first embodiment.

To displace the retaining member 104 off of the outer circumference 106 of the needle holder 102 and about the reduced diameter 112 of the needle holder 102, the piston 16, and more particularly, the piston seal 114 may have a punch 116 formed about a distal end of the piston seal 114, as shown in FIG. 6. In particular, as discussed above, the distal end of the piston seal 114 may have the inner ring 118 and the outer ring 120 which defines the annular suction groove 122. The punch 116 may have an annular configuration and extend beyond the depth of the inner and outer rings 118, 120. The punch 116 may be sized, configured and positioned on the distal end of the piston seal 114 so as to mate with an upper surface 124 (see FIG. 7) of the retaining member 104. When the piston 16 is traversed from the retracted position to a first extended position (see FIG. 11), a distal tip of the punch 116 initially contacts the upper surface 124 (see FIG. 7) of the retaining member 104. As the user continues to depress the thumb platform 60 to traverse the piston 16 to a second extended position (see FIG. 8), the punch 116 displaces the retaining member 104 off of the outer circumference 106 and about the reduced diameter 112 of the needle holder 102. In this instance, after fluid is introduced into the variable fluid chamber 20 and expelled through the needle 12 and into the patient, the suction force of the suction groove 122 may be greater than any frictional force between the needle holder 102 and the body 14 and/or retaining member 104. Accordingly, the retraction force of the variable vacuum compartment 36 urges the piston 16 toward the retracted position and the suction force draws the needle holder 102 and needle 12 within the body 14 of the safety syringe 100 after the medical professional has released the thumb platform 60, as shown in FIG. 9.

As used in relation to the second embodiment of the syringe 100, the first extended position describes the piston's position when the distal end of the punch 116 contacts the upper surface 124 of the retaining member 102 and the retaining member 102 is disposed about the outer circumference 106 of the needle holder 102, as shown in FIG. 11. Also, the second extended position describes the piston's position when the punch 116 of the piston 16 has displaced the retaining member 104 off of the outer circumference 106 and about the reduced diameter 112, as shown in FIG. 8. The retracted position has the same definition as the retracted position as defined in relation to the first embodiment of the syringe 10, as shown in FIGS. 6 and 9.

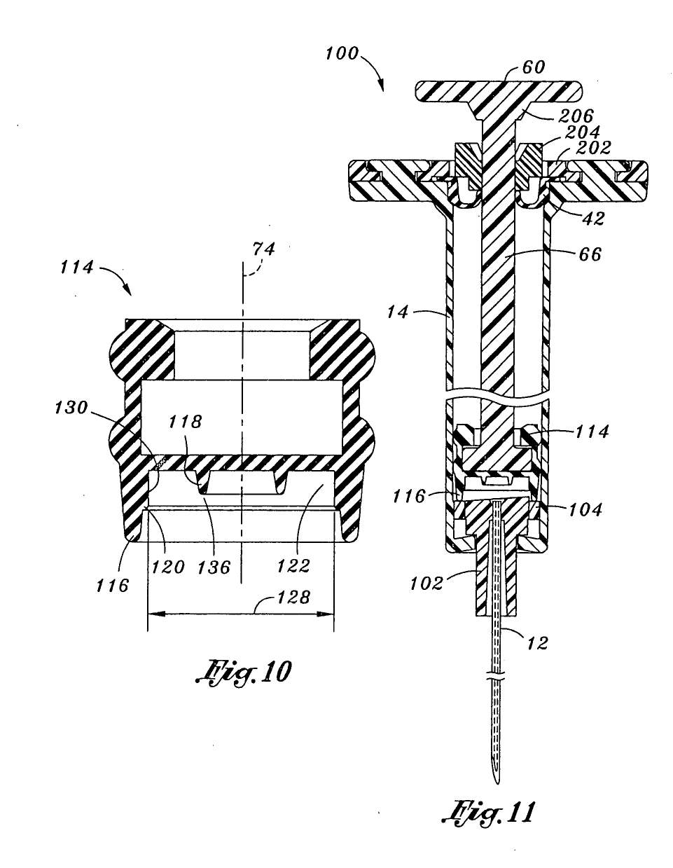

In use, the second embodiment of the safety syringe 100 may be provided to the medical professional or user with the piston 16 in the retracted position (see FIG. 6) without any fluid in the variable fluid chamber 20. To fill the variable fluid chamber 20 of the safety syringe 100 with medication, the medical professional may depress the thumb platform 60 so as to traverse the piston 16 toward or to the first extended position (see FIG. 11). At the first extended position, the distal end of the piston 16 does not create a suction force with the retaining member 104 and the top surface 126 (see FIG. 7) of the needle holder 102. In particular, the upper surface 124 of the retaining member 104 and/or the top surface 126 of the needle holder 102 may have a textured surface similar to the textured top surface 58 of the needle holder 18 of the first embodiment of the safety syringe 10. The textured upper surface 124 of the retaining member 104 permits air to enter into a pocket 136 (see FIGS. 6 and 10) to prevent creation of any suction force in the absence of fluid in the variable fluid chamber 20. Further, the textured top surface 126 of the needle holder 102 permits air to enter into the suction groove 122 to prevent creation of any suction force in the event that the inner and outer rings 118, 120 were to contact the top surface 126 of the needle holder 102.

With the piston 16 at the first extended position (see FIG. 11), the medical professional may insert the needle 12 of the safety syringe 100 into a medication container filled with fluidic medication. The medical professional may slowly cause the piston 16 to traverse back toward the retracted position by reducing the thumb pressure applied to the thumb platform 60 until the retraction force is greater than the thumb pressure applied to the thumb platform 60. As the piston 16 is traversed back toward the retracted position (see FIG. 6), the fluidic medication in the medication container is transferred into the variable fluid chamber 20 of the safety syringe 100 via the needle 12. After the correct amount of fluidic medication is transferred into the variable fluid chamber 20, the medical professional removes the needle 12 from the medication container and inverts the syringe 100 to prepare to remove any residual air within the variable fluid chamber 20.

With the safety syringe 100 inverted, the medical professional may tap the outer surface of the body 14 to urge any air bubbles within the variable fluid chamber 20 toward the needle 12. The medical professional then slightly depresses the thumb platform 60 to expel any residual air within the variable fluid chamber 20 to the environment. The safety syringe 100 has now been prepared for administrating the fluidic medication to the patient.

The medical professional may now insert the needle 12 into a skin of a patient and traverse the piston 16 toward the first extended position (see FIG. 11). When the piston 16 is at the first extended position, a majority of the fluidic medication is now transferred from the variable fluid chamber 20 to the patient. The medical professional may then further depress the thumb platform 60 to traverse the piston 16 from the first extended position to the second extended position (see FIG. 8).

At the second extended position, the punch 116 displaces the retaining member 104 off of the outer circumference 106 of the needle holder 102 and about the reduced diameter 112 of the needle holder 102. Simultaneously or at about the same time, the annular suction groove 122 creates a suction force on the top surface 126 of the needle holder 102 due to the surface tension of the fluid on the top surface 126 of the needle holder 102 and the inner and outer rings 118, 120. Additionally, surface tension between the inner surface 130 of the outer ring 120 and the outer circumference 106 of the needle holder may create a suction force so as to engage the piston 16 and the needle holder 102. After the fluidic medication is completely injected into the patient, the medical professional may remove the needle 12 from the patient and release the thumb platform 60 to automatically retract the needle holder 102 and needle 12 into the body 14 of the safety syringe 100 thereby protecting the medical professional and patient and other personnel from accidental needle prickings and needle reuse. In particular, when the piston 16 is traversed to the second extended position, the variable vacuum compartment 36 creates the retraction force which is greater than any frictional force between the needle holder 102 and the body 14 of the safety syringe 100. When the thumb platform 60 is released, the retraction force urges the piston 16 to the retracted position. The suction force between the piston 16 and the needle holder 102 urges the needle holder 102 and the needle 12 into the syringe body 14 due to the traversal of the piston 16 to the retracted position.

In the second embodiment of the safety syringe 100, the needle holder 102 may not have a ring groove 44 nor a first seal 40 disposed within the ring groove 44. Rather, as discussed above, the needle holder 102 of the second embodiment of the safety syringe 100 may define an outer circumference 106 and a reduced lower diameter 112. Moreover, in the second embodiment of the safety syringe 100, as shown in FIG. 10, the piston seal 114 may further have a punch 116 formed at the distal end of the piston seal 114 about the outer ring 120. The inner ring 118 of the piston seal 114 may have a similar configuration as the first embodiment of the safety syringe 10. The outer ring 120 of the piston seal 114 may have an inner diameter 128 defining an inner surface 130 which may be parallel to the central axis 74 of the safety syringe 100. Also, the inner diameter 128 may be about equal to an outer diameter 134 of the outer circumference 106 of the needle holder 102. Moreover, the outer ring 120 may extend beyond the depth of the inner ring 118. The difference in depth between the inner ring 118 and the outer ring 120 may create the pocket 136 in which the outer circumference 106 of the upper portion of the needle holder 102 may be inserted into when the piston 16 is extended to the second extended position (see FIG. 11). The outer ring 120 may surround the upper portion of the needle holder 102.

The distal end of the piston seal 114 may have a punch 116. The punch 116 may be sufficiently rigid so as to apply a downward force onto the retaining member 104 to displace the retaining member off of the outer circumference 106 and about the lower reduced diameter 112. The punch 116 may further be lined with an outer plastic cap to further add rigidity to the punch 116 and yet retain the resiliency and softness of the piston seal 114. The outer cap may be disposed about the distal end of the punch 116. When the piston 16 is traversed to the second extended position, the outer surface of the outer cap directly contacts the upper surface 124 of the retaining member 104 and pushes the retaining member 104 off of the outer circumference 106 and about the reduced diameter 112.

In both the first and second embodiments of the safety syringe 10, 100, the needle may be canted to one side of the syringe body 14 when the needle 12 is retracted into the syringe body 14 (see FIGS. 5 and 9). To this end, the textured top surface 58, 126 may be uneven (i.e., not parallel) with the annular suction groove 38, 122, as shown in FIGS. 3a and 7. For example, the annular suction groove 38, 122 may be angularly offset 80, 138 from the textured top surface 58, 126 about four (4) degrees, as shown in FIGS. 3a and 7. More particularly, the annular suction groove 38, 122 may be perpendicular with a central axis 74 of the syringe body 14, whereas the textured top surface 58, 126 may be about eighty six (86) degrees offset with respect to the central axis 74 of the syringe body 14 (see FIGS. 1 and 3a and 7) or four (4) degrees with respect to a transverse plane of the central axis 74. When the needle 12 is retracted into the syringe body 14, the needle 12 is also canted about four degrees toward the syringe body 14 (see FIG. 5 and 9). Now that the needle 12 is canted to one side, the needle 12 is retained within the syringe body 14. For example, if the piston 16 was to be re-traversed toward the extended position (first embodiment) or the first or second extended positions (second embodiment), a tip 78 of the needle 12 would bump into needle stops 76 (see FIG. 5) which would prevent the needle 12 from escaping out of the syringe body 14.

In an aspect of the safety syringe 10, 100 of the first and second embodiments, the same may have an optional braking mechanism. The optional braking mechanism may be a plunger lock 62 as shown in relation to the first embodiment of the safety syringe 10 or have structure similar to the braking mechanism described in U.S. Provisional Patent Application No. 60/679,113, the entire contents of which are expressly incorporated herein by reference. When the syringe 10, 100 is in use, but for the optional braking mechanism and thumb pressure, the retraction force of the variable vacuum compartment 36 would retract the piston 16 into the syringe body 14.

Referring now to the plunger lock shown in FIGS. 1, 4 and 5, the plunger lock 62 may be integrated or attached to a finger platform 64 at the proximal end of the body 14, as shown in FIG. 1. Although the plunger lock 62 will be discussed in relation to the first embodiment of the safety syringe 10, the plunger lock 62 may also be employed in the second embodiment of the safety syringe 100. The plunger lock 62 may be an elongate member 68 which extends upward and against an outer surface 72 of a rigid shaft 66 of the plunger 22 in the direction of arrow A shown in FIG. 1. The distal end 70 of the elongate member 68 may be biased against the outer surface 72 of the rigid shaft 66 and creates a friction force therebetween which is greater than the retraction force created by the variable vacuum compartment 36 at the first extended position. To release the plunger lock 62 from the plunger 22, the user may push the elongate member 68 such that the distal end 70 of the elongate member 68 does not fully engage the outer surface 72 of the rigid shaft 66. In this manner, the frictional force created by the plunger lock 62 is now less than the retraction force of the variable vacuum compartment 36 and the retraction force is capable of retracting the needle holder 18 and needle 12 into the body 14.

Referring now to the braking mechanism shown in FIGS. 6, 8, 9, 11 and 12 the braking mechanism 200 described in the '113 application may be disposed about the proximal end of the body 14 of the safety syringe 100. Although the braking mechanism 200 will be discussed in relation to the second embodiment of the safety syringe 100, the braking mechanism 200 may also be employed in the first embodiment of the safety syringe 10. The braking mechanism 200 may comprise an attachment base 202, shaft brake 204 and a ram member 206. The attachment base 202 may be engaged to the proximal end of the body 14. The attachment base 202 together with the second seal 42 forms a watertight and airtight seal between the proximal end of the body 14 and the shaft 66 of the plunger 22. The plunger 22 may be received through a central aperture of the attachment base 202 and be able to traverse through the aperture of the attachment base 202 without releasing or introducing air into the variable vacuum compartment 36. Finger platforms 64 may collectively have a cavity sized and configured to receive the attachment base 202. A cavity may also be formed in the attachment base 202 which is sized and configured to receive an attachment prong of the shaft brake 204. The shaft brake 204 may be engaged to the attachment base 202 by engaging the attachment prong of the shaft brake 204 into the cavity of the attachment base 202. When the shaft brake 204 is received into the attachment base's cavity, the shaft brake 204 is held securely to the attachment base 202.

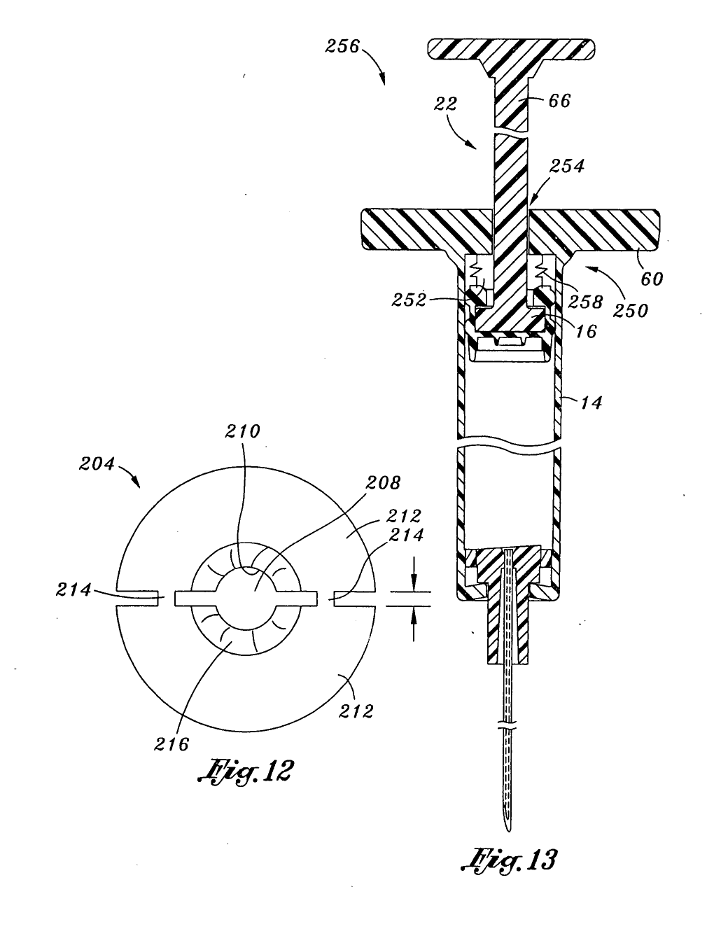

As shown in FIG. 12, the shaft brake 204 may also have a central aperture 208 through which the plunger 22 is traversably disposed. The aperture 208 of the shaft brake 204 may have a diameter sized to the outer diameter of the shaft 66 so as to create a friction fit therebetween. The frictional forces between the inner surface 210 of the shaft brake aperture 208 and the outer surface 72 of the rigid shaft 66 of the plunger 22 may be greater than the retraction force of the variable vacuum compartment 36 when the piston 16 is disposed at the extended position (first embodiment) or the first extended position or the second extended position (second embodiment). In this manner, whenever the shaft brake 204 is engaged to the shaft 66 of the plunger 22, the piston 16 is not moveable or is only negligibly traversable within the body 14 during the operation of filling the variable fluid chamber 20 with fluidic medication or injecting the patient with the fluidic medication contained within the variable fluid chamber 20.

To disengage the shaft brake 204 from the rigid shaft 66 of the plunger 22, the ram member 206 attached to a bottom surface of the thumb platform 60 spreads the shaft brake 204 apart such that the inner surface 210 of the shaft brake aperture 208 does not frictionally engage the outer surface 72 of the rigid shaft 66. In particular, as shown in FIG. 12, a top view of the shaft brake 204 shows that the shaft brake 204 may be formed by two half discs 212 joined by a hinge element 214. When the hinge element 214 is intact with the two half discs 212, the inner surface 210 of the shaft brake aperture 208 frictionally engages the outer surface 72 of the rigid shaft 66. A central portion of the shaft brake 204 may have a frustal-conical inner surface 216 (see FIGS. 6, 9 and 12). This frustal-conical inner surface 216 mates with a frustal-conical outer surface 218 of the ram member 206 (see FIGS. 8 and 9). When the thumb platform 60 is traversed downward, the frustal-conical outer surface 218 mates with the frustal-conical inner surface 216. When the thumb platform 60 is further depressed, the frustal-conical outer surface 218 applies a radially outward force on the frustal-conical inner surface 216. This radial outward force urges the two half discs 212 apart and ultimately breaks the hinge element 214 thereby disengaging the outer surface 72 of the rigid shaft 66 and the inner surface 210 of the shaft brake aperture 208. The hinge element 214 may be broken at about the same time that the piston 16 displaces the retaining member 104 off of the outer circumference 106 and about the reduced lower diameter 112 (i.e., piston 16 in second extended position). With the hinge element 214 broken, the retraction force of the variable vacuum compartment 36 may draw the needle 12 into the body 14 upon engagement between the piston 16 and the needle holder 102.

In another aspect of the safety syringe 10, 100, the retraction force of the variable vacuum compartment 36 may be created by a spring mechanism 250, as shown in FIG. 13. In particular, the cavity 252 between the proximal end of the body 14 and the piston 16 within the body 14 may be vented to the environment. As such, when the piston 16 is traversed toward the extended position (first embodiment) or the first or second extended positions (second embodiment), air molecules are introduced into such cavity 252. In FIG. 13, the air molecules are introduced into the cavity 252 via a gap 254 between the rigid shaft 66 of the plunger 22 and an aperture of the thumb platform 60. The safety syringe 256 shown in FIG. 13 may be operated in a similar fashion with respect to the second embodiment of the safety syringe 100 with or without the braking mechanism. Although the spring mechanism is shown in relation to the second embodiment of the safety syringe 100, it is contemplated that the various aspects of the spring mechanism 250 may also be employed in the first embodiment of the safety syringe 10.

The spring mechanism 250 may comprise at least one tension spring 258. Preferably, as shown in FIG. 13, the spring mechanism 250 may have an even number (e.g., two) of tension springs 258 to balance retraction of the piston 16 toward the retracted position. By way of example and not limitation, the tension spring 258 may be a helical spring designed for tension or an elongate elastic material, etc. A proximal end of the tension spring 258 may be attached to the proximal end of the body 14. Also, a distal end of the tension spring 258 may be attached to the piston 16. When the piston is at the retracted position, the tension spring 258 may be relaxed. As the piston 16 is traversed toward the extended position or the first and second extended positions, the tension spring 258 may come under tension thereby urging the piston 16 back toward the retracted position and defining a retraction force.

The above description is given by way of example and not limitation. Given the above disclosure, one skilled in the art could devise variations that are within the scope and spirit of the invention disclosed herein. For example, the dimensions and other ranges provided above are for the purpose of illustration and other sizes and proportions may be employed. Further, the various features of the embodiment disclosed herein can be used alone, or in varying combinations with each other and are not intended to be limited to the specific combination described herein. Thus, the scope of the claims is not to be limited by the illustrated embodiment.

What is claimed is:

1. A retractable safety syringe apparatus, comprising:
a syringe body defining a distal end and a proximal end;
a plunger having a piston slideably disposed within the syringe body and a shaft extending through the proximal end, a distal end surface of the piston having a suction depression for forming a fluid activated suction force;
a distal seal disposed between the piston and the syringe body for forming a fluid tight seal between the plunger piston and the syringe body;
a needle holder removably engaged to the distal end of the syringe body, the needle holder defining a textured top surface engagable to the suction depression of the piston in the presence of fluid; and
a needle attached to the needle holder and extending out from a distal end of the syringe body.
wherein surface tension of the fluid and the suction groove and the top surface of the needle holder creates the suction force which retracts the needle holder into the syringe body.

2. The syringe of claim 1 wherein the suction depression is an annular suction groove.

3. The syringe of claim 2 wherein the suction groove is defined by an inner ring and an outer ring disposed on a distal end of the piston.

4. The syringe of claim 1 further comprising a proximal seal disposed between the shaft and the syringe body for forming an airtight seal between the plunger shaft and the syringe body, wherein the proximal seal, distal seal and the syringe body define a variable vacuum compartment which produces a retraction force when the piston is traversed to the extended position.

5. The syringe of claim 4 wherein the extended position is a first extended position or a second extended position.

6. The syringe of claim 1 wherein the suction depression is a pocket.

7. The syringe of claim 6 wherein the pocket is defined by an outer ring having an inner diameter equal to about an outer diameter of the needle holder.

8. The syringe of claim 1 further comprising a tension member attached to the proximal end of the body and the piston for creating a retraction force when the piston is traversed toward an extended position.

9. The syringe of claim 1 wherein a top surface of the needle holder is skewed with respect to a central axis of the body for canting the needle when the needle is retracted into the body.

10. The syringe of claim 1 further comprising a plunger lock attached to the proximal end of the body, the plunger lock resisting traversal of the plunger due to a retraction force, the plunger lock comprising an elongate member frictionally engaged to an outer surface of the shaft.

11. The syringe of claim 1 further comprising braking mechanism having a ram member attached to the plunger and a shaft brake frictionally engaged to the plunger, the ram member having an outer frusto conical surface which mates with an inner frusto conical surface of the shaft brake, the outer frusto conical surface of the ram member being operative to disengage the shaft brake from the shaft.
